(19) United States
(12) Patent Application Publication
(10) Pub. No.: US 2026/0345788 A1
(43) Pub. Date: Nov. 5, 2026

(54) NUCLEIC ACID COMPLEXES AND USES THEREOF

(71) Applicant: BioNTech SE, Mainz (DE)

(72) Inventors: Jorge Moreno Herrero, Mainz (DE); Heinrich Haas, Mainz (DE); Stephanie Erbar, Chiclana de la Frontera (ES); Theo Benjamin Stahl, Mainz (DE)

(21) Appl. No.: 19/122,492

(22) PCT Filed: Oct. 20, 2023
(86) PCT No.: PCT/IB2023/060639

§ 371 Date: Apr. 18, 2025

(30) Foreign Application Priority Data
Oct. 21, 2022 (WO) PCT/IB22/60153

(51) Int. Cl. A61K 47/54; A61K 47/59; A61K 47/64

(52) U.S. Cl. CPC A61K 47/549; A61K 47/59; A61K 47/645

(57) ABSTRACT

The present disclosure provides complexes and uses thereof, wherein the complex comprises a first cationic polymer, a second cationic polymer, and a monomeric RNA molecule, wherein the first cationic polymer comprises greater than 250 repeating monomer units, and the second cationic polymer comprises 200 repeating monomer units, or less.

### CROSS REFERENCE TO RELATED APPLICATIONS

This application claims priority to and benefit of International Patent Application No. PCT/IB22/60153, filed Oct. 21, 2022, the entire contents of which are hereby incorporated by reference herein.

### BACKGROUND

Use of nucleic acids, e.g., RNA, has emerged as a means for delivering therapy in recent years. Nucleic acid therapies have shown particular success in the space of infectious disease vaccines, and have exhibited promise as cancer immunotherapies, therapeutic protein replacement therapies, and treatment of genetic diseases. Delivering nucleic acid therapy to a cellular target faces certain challenges, as RNA is unstable and prone to degradation by nucleases. See Wadhwa, et al., Pharmaceutics, 12(2): 102 (2020).

### SEQUENCE LISTING

The present specification makes reference to a Sequence Listing (submitted electronically as a .xml file named 2013237-0465_SL.XML) on Oct. 21, 2022. The .xml file was generated on Oct. 18, 2022, and is 6 kilobytes in size. The entire contents of the Sequence Listing are herein incorporated by reference.

### SUMMARY

There remains a need for delivery vehicles of nucleic acids, e.g., RNA, that are capable of withstanding the conditions in the body, while effectively and efficiently delivering the nucleic acid to a target cell, without requiring substantial amounts of the nucleic acid to achieve such effectiveness. Cationic polymers have been recognized as useful for developing such delivery vehicles, as reported in PCT App. Pub. No. WO 2021/001417, the entirety of which is incorporated herein by reference. High molecular weight cationic polymers, while useful for the delivery of nucleic acids, exhibit dose-limiting toxicities that limit the dose range available to achieve a therapeutic effect. Moreover, high molecular weight cationic polymers can exhibit particularly strong binding affinity to nucleic acid molecules, and thereby inhibit the release of the nucleic acid after administration to the patient, again limiting potential therapeutic window.

It has been discovered, however, that complexes comprising mixtures of high molecular weight cationic polymers and low molecular weight cationic polymers not only avoid the problems associated with complexes comprising only high molecular weight cationic polymers, but further are capable of forming smaller sized particles capable of delivering monomeric RNA to a target.

The present disclosure provides, among other things, complexes for delivery nucleic acids, e.g., RNA, that exhibit certain improvements over previous complex formulations. In some embodiments, the present disclosure provides a complex comprising a first cationic polymer, a second cationic polymer, and a monomeric RNA molecule, wherein the first cationic polymer comprises greater than 250 repeating monomer units, and the second cationic polymer comprises 200 repeating monomer units, or less.

In some embodiments, the present disclosure provides a method of increasing or causing increased expression of RNA in a target in a subject comprising administering to the subject a complex described herein.

In some embodiments, the present disclosure provides a method of treating a disease, disorder, or condition in a subject comprising administering to the subject a complex described herein.

In some embodiments, the present disclosure provides a complex described herein, for use as a medicament.

In some embodiments, the present disclosure provides a complex described herein, for use in the treatment and/or prevention of a disease or disorder, wherein the disease or disorder is an infectious disease, cancer, a genetic disorder, an autoimmune disease, or a rare disease.

### BRIEF DESCRIPTION OF THE DRAWING

FIG. 1A is a plot illustrating the average hydrodynamic diameter of formulated with saRNA or modRNA at different PEI-mTAT mixing ratios.

FIG. 1B is a plot illustrating luciferase expression of complexes formulated with saRNA or modRNA at different PEI-mTAT mixing ratios in C2C12 cells and a 100 ng dose.

FIG. 2A is a plot illustrating the average hydrodynamic diameter of complexes comprising modRNA and having different PEI-pArg mixing ratios and pArg lengths.

FIG. 2B is a plot illustrating the average hydrodynamic diameter of complexes comprising saRNA and having different PEI-pArg mixing ratios and pArg lengths.

FIG. 2C is a plot illustrating the luciferase expression of complexes comprising modRNA and having different PEI-pArg mixing ratios and pArg lengths in C2C12 cells at a 100 ng dose.

FIG. 2D is a plot illustrating the luciferase expression of complexes comprising saRNA having different PEI-pArg mixing ratios and pArg lengths in C2C12 cells at a 100 ng dose.

FIG. 3A is a plot illustrating luciferase expression for the tested complexes over the course of the experiment with formulated saRNA at different PEI-mTAT ratios.

FIG. 3B is a plot illustrating the area under the curve of luciferase expression over the whole duration of the experiment with formulated saRNA at different PEI-mTAT mixing ratios.

FIG. 3C is a plot illustrating luciferase expression for the tested complexes over time.

FIG. 3D is a plot illustrating the area under the curve of luciferase expression over time with tested complexes.

FIG. 3E is a plot illustrating the average hydrodynamic diameter of the tested complexes.

### DETAILED DESCRIPTION OF CERTAIN EMBODIMENTS

The present disclosure provides, among other things, complexes useful for delivery of a nucleic acid, e.g., RNA, and uses of such complexes. In some embodiments, such complexes exhibit improved properties over previous formulations, such as small particle size, complexation with monomeric RNA molecules, and improved biological activity. In some embodiments, complexes described herein are useful for the treatment of a variety of diseases. In some embodiments, such complexes can be administered via parenteral, systemic, intravenous, or intranasal means.

### DEFINITIONS

Complexes of this disclosure include those described generally above and are further illustrated by the classes, subclasses, and species disclosed herein. As used herein, the following definitions shall apply unless otherwise indicated. For purposes of this disclosure, the chemical elements are identified in accordance with the Periodic Table of Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

Unless otherwise stated, structures depicted herein are meant to include all stereoisomeric (e.g., enantiomeric or diastereomeric) forms of the structure, as well as all geometric or conformational isomeric forms of the structure. For example, the R and S configurations of each stereocenter are contemplated as part of the disclosure. Therefore, single stereochemical isomers, as well as enantiomeric, diastereomeric, and geometric (or conformational) mixtures of provided compounds are within the scope of the disclosure. Unless otherwise stated, all tautomeric forms of provided compounds are within the scope of the disclosure.

Unless otherwise indicated, structures depicted herein are meant to include compounds that differ only in the presence of one or more isotopically enriched atoms. For example, compounds having the present structures including replacement of hydrogen by deuterium or tritium, or replacement of a carbon by ¹³C- or ¹⁴C-enriched carbon are within the scope of this disclosure.

About or approximately: As used herein, the term “approximately” or “about,” as applied to one or more values of interest, refers to a value that is similar to a stated reference value. In general, those skilled in the art, familiar within the context, will appreciate the relevant degree of variance encompassed by “about” or “approximately” in that context. For example, in some embodiments, the term “approximately” or “about” may encompass a range of values that are within (i.e., ±) 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administering: As used herein, the term “administering” or “administration” typically refers to the administration of a composition to a subject to achieve delivery of an agent that is, or is included in, a composition to a target site or a site to be treated. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may be parenteral. In some embodiments, administration may be oral. In some particular embodiments, administration may be intravenous. In some particular embodiments, administration may be subcutaneous. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, administration may comprise a prime-and-boost protocol. A prime-and-boost protocol can include administration of a first dose of a pharmaceutical composition (e.g., an immunogenic composition, e.g., a vaccine) followed by, after an interval of time, administration of a second or subsequent dose of a pharmaceutical composition (e.g., an immunogenic composition, e.g., a vaccine). In the case of an immunogenic composition, a prime-and-boost protocol can result in an increased immune response in a patient.

Aliphatic: The term “aliphatic” refers to a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point or more than one points of attachment to the rest of the molecule. Unless otherwise specified, aliphatic groups contain 1-12 aliphatic carbon atoms. As used herein, it is understood that an aliphatic group can also be bivalent (e.g., encompass a bivalent hydrocarbon chain that is saturated or contains one or more units of unsaturation, such as, for example, —CH₂—, —CH₂—CH₂—, —CH₂—CH₂—CH₂—, and so on). In some embodiments, aliphatic groups contain 1-6 aliphatic carbon atoms (e.g., C₁₋₆). In some embodiments, aliphatic groups contain 1-5 aliphatic carbon atoms (e.g., C₁₋₅). In other embodiments, aliphatic groups contain 1-4 aliphatic carbon atoms (e.g., C₁₋₄). In still other embodiments, aliphatic groups contain 1-3 aliphatic carbon atoms (e.g., C₁₋₃), and in yet other embodiments, aliphatic groups contain 1-2 aliphatic carbon atoms (e.g., C₁₋₂). In some embodiments, “cycloaliphatic” refers to a monocyclic C₃₋₈ hydrocarbon or a bicyclic C₇₋₁₀ hydrocarbon that is completely saturated or that contains one or more units of unsaturation, but which is not aromatic, that has a single point or more than one points of attachment to the rest of the molecule. Suitable aliphatic groups include, but are not limited to, linear or branched, substituted or unsubstituted alkyl, alkenyl, or alkynyl groups and hybrids thereof. A preferred aliphatic group is C₁₋₆ alkyl.

Alkyl: The term “alkyl”, used alone or as part of a larger moiety, refers to a saturated, optionally substituted straight or branched chain hydrocarbon group having (unless otherwise specified) 1-12, 1-10, 1-8, 1-6, 1-4, 1-3, or 1-2 carbon atoms (e.g., C₁₋₁₂, C₁₋₁₀, C₁₋₈, C₁₋₆, C₁₋₄, C₁₋₃, or C₁₋₂). Exemplary alkyl groups include methyl, ethyl, propyl, butyl, pentyl, hexyl, and heptyl.

Alkenyl: The term “alkenyl”, used alone or as part of a larger moiety, refers to an optionally substituted straight or branched chain or cyclic hydrocarbon group having at least one double bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkenyl groups include ethenyl, propenyl, butenyl, pentenyl, hexenyl, and heptenyl. The term “cycloalkenyl” refers to an optionally substituted non-aromatic monocyclic or multicyclic ring system containing at least one carbon-carbon double bond and having about 3 to about 10 carbon atoms. Exemplary monocyclic cycloalkenyl rings include cyclopentenyl, cyclohexenyl, and cycloheptenyl.

Alkynyl: The term “alkynyl”, used alone or as part of a larger moiety, refers to an optionally substituted straight or branched chain hydrocarbon group having at least one triple bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkynyl groups include ethynyl, propynyl, butynyl, pentynyl, hexynyl, and heptynyl.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Aryl: The term “aryl” refers to monocyclic and bicyclic ring systems having a total of five to fourteen ring members (e.g., C₅-C₁₄), wherein at least one ring in the system is aromatic and wherein each ring in the system contains three to seven ring members. In some embodiments, an “aryl” group contains between six and twelve total ring members (e.g., C₆-C₁₂). The term “aryl” may be used interchangeably with the term “aryl ring”. In certain embodiments of the present invention, “aryl” refers to an aromatic ring system which includes, but not limited to, phenyl, biphenyl, naphthyl, anthracyl and the like, which may bear one or more substituents. Unless otherwise specified, “aryl” groups are hydrocarbons. In some embodiments, an “aryl” ring system is an aromatic ring (e.g., phenyl) that is fused to a non-aromatic ring (e.g., cycloalkyl). Examples of aryl rings include that are fused include

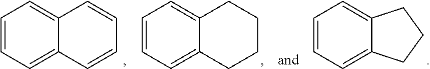

Associated: Two events or entities are “associated” with one another, as that term is used herein, if the presence, level and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biological sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids (e.g., sperm, sweat, tears), secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example, nucleic acids or proteins extracted from a sample or obtained by subjecting a primary sample to techniques such as amplification or reverse transcription of mRNA, isolation and/or purification of certain components, etc.

Carrier: As used herein, the term “carrier” refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Combination therapy: As used herein, the term “combination therapy” refers to those situations in which a subject is simultaneously exposed to two or more therapeutic regimens (e.g., two or more therapeutic agents or modality (ies)). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality (ies) to a subject receiving the other agent(s) or modality (ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Cycloaliphatic: As used herein, the term “cycloaliphatic” refers to a monocyclic C₃₋₈ hydrocarbon or a bicyclic C₇₋₁₀ hydrocarbon that is completely saturated or that contains one or more units of unsaturation, but which is not aromatic, that has a single point or more than one points of attachment to the rest of the molecule.

Cycloalkyl: As used herein, the term “cycloalkyl” refers to an optionally substituted saturated ring monocyclic or polycyclic system of about 3 to about 10 ring carbon atoms. Exemplary monocyclic cycloalkyl rings include cyclopropyl, cyclobutyl, cyclopentyl, cyclohexyl, and cycloheptyl.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen or therapeutic regimen: Those skilled in the art will appreciate that the terms “dosing regimen” and “therapeutic regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Excipient: As used herein, the term “excipient” refers to a non-therapeutic agent that may be included in a pharmaceutical composition, for example, to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Heteroaliphatic: The term “heteroaliphatic” or “heteroaliphatic group”, as used herein, denotes an optionally substituted hydrocarbon moiety having, in addition to carbon atoms, from one to five heteroatoms, that may be straight-chain (i.e., unbranched), branched, or cyclic (“heterocyclic”) and may be completely saturated or may contain one or more units of unsaturation, but which is not aromatic. The term “heteroatom” refers to nitrogen, oxygen, or sulfur, and includes any oxidized form of nitrogen or sulfur, and any quaternized form of a basic nitrogen. The term “nitrogen” also includes a substituted nitrogen. Unless otherwise specified, heteroaliphatic groups contain 1-10 carbon atoms wherein 1-3 carbon atoms are optionally and independently replaced with heteroatoms selected from oxygen, nitrogen, and sulfur. In some embodiments, heteroaliphatic groups contain 1-4 carbon atoms, wherein 1-2 carbon atoms are optionally and independently replaced with heteroatoms selected from oxygen, nitrogen, and sulfur. In yet other embodiments, heteroaliphatic groups contain 1-3 carbon atoms, wherein 1 carbon atom is optionally and independently replaced with a heteroatom selected from oxygen, nitrogen, and sulfur. Suitable heteroaliphatic groups include, but are not limited to, linear or branched, heteroalkyl, heteroalkenyl, and heteroalkynyl groups. For example, a 1- to 10 atom heteroaliphatic group includes the following exemplary groups: —O—CH₃, —CH₂—O—CH₃, —O—CH₂—CH₂—O—CH₂—CH₂—O—CH₃, and the like.

Heteroaryl: The terms “heteroaryl” and “heteroar-”, used alone or as part of a larger moiety, e.g., “heteroaralkyl”, or “heteroaralkoxy”, refer to monocyclic or bicyclic ring groups having 5 to 12 ring atoms (e.g., 5- to 6-membered monocyclic heteroaryl or 9- to 12-membered bicyclic heteroaryl); having 6, 10, or 14 π-electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, imidazo[1,2-a]pyrimidinyl, imidazo[1,2-a]pyridyl, imidazo[4,5-b]pyridyl, imidazo[4,5-c]pyridyl, pyrrolopyridyl, pyrrolopyrazinyl, thienopyrimidinyl, triazolopyridyl, and benzoisoxazolyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring (i.e., a bicyclic heteroaryl ring having 1 to 3 heteroatoms). Nonlimiting examples include indolyl, isoindolyl, benzothienyl, benzofuranyl, dibenzofuranyl, indazolyl, benzimidazolyl, benzotriazolyl, benzothiazolyl, benzothiadiazolyl, benzoxazolyl, quinolyl, isoquinolyl, cinnolinyl, phthalazinyl, quinazolinyl, quinoxalinyl, 4H quinolizinyl, carbazolyl, acridinyl, phenazinyl, phenothiazinyl, phenoxazinyl, tetrahydroquinolinyl, tetrahydroisoquinolinyl, pyrido[2,3-b]-1,4-oxazin-3 (4H)-one, 4H-thieno[3,2-b]pyrrole, and benzoisoxazolyl. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

Heteroatom: The term “heteroatom” as used herein refers to nitrogen, oxygen, or sulfur, and includes any oxidized form of nitrogen or sulfur, and any quaternized form of a basic nitrogen.

Heterocycle: As used herein, the terms “heterocycle”, “heterocyclyl”, “heterocyclic radical”, and “heterocyclic ring” are used interchangeably and refer to a stable 3- to 8-membered monocyclic, a 7- to 12-membered bicyclic, or a 10- to 16-membered polycyclic heterocyclic moiety that is either saturated or partially unsaturated, and having, in addition to carbon atoms, one or more, such as one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or NR⁺ (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, azetidinyl, oxetanyl, tetrahydrofuranyl, tetrahydrothienyl, pyrrolidinyl, piperidinyl, decahydroquinolinyl, oxazolidinyl, piperazinyl, dioxanyl, dioxolanyl, diazepinyl, oxazepinyl, thiazepinyl, morpholinyl, and thiamorpholinyl. A heterocyclyl group may be mono-, bi-, tri-, or polycyclic, preferably mono-, bi-, or tricyclic, more preferably mono- or bicyclic. The term “heterocyclylalkyl” refers to an alkyl group substituted by a heterocyclyl, wherein the alkyl and heterocyclyl portions independently are optionally substituted. A bicyclic heterocyclic ring also includes groups in which the heterocyclic ring is fused to one or more aryl rings. Exemplary bicyclic heterocyclic groups include indolinyl, isoindolinyl, benzodioxolyl, 1,3-dihydroisobenzofuranyl, 2,3-dihydrobenzofuranyl, tetrahydroquinolinyl,

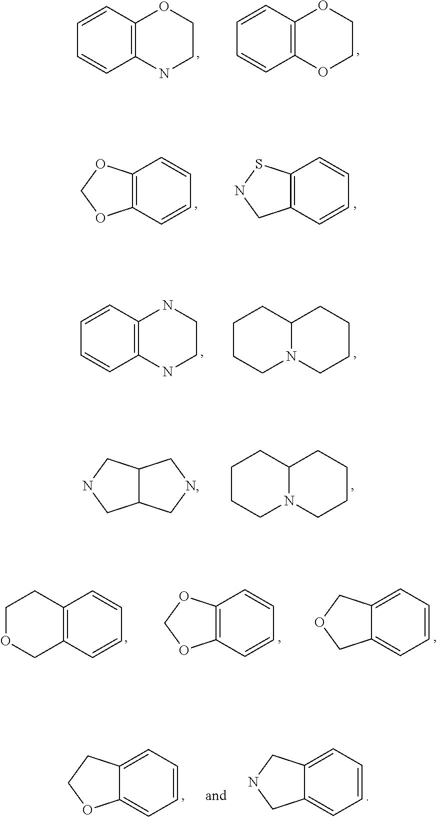

A bicyclic heterocyclic ring can also be a spirocyclic ring system (e.g., 7- to 11-membered spirocyclic fused heterocyclic ring having, in addition to carbon atoms, one or more heteroatoms as defined above (e.g., one, two, three or four heteroatoms)). A bicyclic heterocyclic ring can also be a bridged ring system (e.g., 7- to 11-membered bridged heterocyclic ring having one, two, or three bridging atoms.

Nucleic acid/Polynucleotide: As used herein, the term “nucleic acid” refers to a polymer of at least 10 nucleotides or more. In some embodiments, a nucleic acid is or comprises DNA. In some embodiments, a nucleic acid is or comprises RNA. In some embodiments, a nucleic acid is or comprises peptide nucleic acid (PNA). In some embodiments, a nucleic acid is or comprises a single stranded nucleic acid. In some embodiments, a nucleic acid is or comprises a double-stranded nucleic acid. In some embodiments, a nucleic acid comprises both single and double-stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long.

Nucleic acid particle: A “nucleic acid particle” can be used to deliver nucleic acid to a target site of interest (e.g., cell, tissue, organ, and the like). A nucleic acid particle may be formed from at least one cationic or cationically ionizable lipid or lipid-like material, at least one cationic polymer such as protamine, or a mixture thereof and nucleic acid. Nucleic acid particles include lipid nanoparticle (LNP)-based and lipoplex (LPX)-based formulations.

Nucleotide: As used herein, the term “nucleotide” refers to its art-recognized meaning. When a number of nucleotides is used as an indication of size, e.g., of a polynucleotide, a certain number of nucleotides refers to the number of nucleotides on a single strand, e.g., of a polynucleotide.

Oral: The phrases “oral administration” and “administered orally” as used herein have their art-understood meaning referring to administration by mouth of a compound or composition.

Parenteral: The phrases “parenteral administration” and “administered parenterally” as used herein have their art-understood meaning referring to modes of administration other than enteral and topical administration, usually by injection, and include, without limitation, intravenous, intramuscular, intra-arterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

Partially unsaturated: As used herein, the term “partially unsaturated” refers to a ring moiety that includes at least one double or triple bond between ring atoms. The term “partially unsaturated” is intended to encompass rings having multiple sites of unsaturation, but is not intended to include aromatic (e.g., aryl or heteroaryl) moieties, as herein defined.

Patient or subject: As used herein, the term “patient” or “subject” refers to any organism to which a provided composition is or may be administered, e.g., for experimental, diagnostic, prophylactic, cosmetic, and/or therapeutic purposes. Typical patients or subjects include animals (e.g., mammals such as mice, rats, rabbits, non-human primates, and/or humans). In some embodiments, a patient is a human. In some embodiments, a patient or a subject is suffering from or susceptible to one or more disorders or conditions. In some embodiments, a patient or subject displays one or more symptoms of a disorder or condition. In some embodiments, a patient or subject has been diagnosed with one or more disorders or conditions. In some embodiments, a patient or a subject is receiving or has received certain therapy to diagnose and/or to treat a disease, disorder, or condition.

Pharmaceutical composition: As used herein, the term “pharmaceutical composition” refers to an active agent, formulated together with one or more pharmaceutically acceptable carriers. In some embodiments, the active agent is present in unit dose amount appropriate for administration in a therapeutic or dosing regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

Polycyclic: As used herein, the term “polycyclic” refers to a saturated or unsaturated ring system having two or more rings (for example, heterocyclyl rings, heteroaryl rings, cycloalkyl rings, or aryl rings), having between 7 and 20 atoms, in which one or more carbon atoms are common to two adjacent rings. For example, in some embodiments, a polycyclic ring system refers to a saturated or unsaturated ring system having three or more rings (for example, heterocyclyl rings, heteroaryl rings, cycloalkyl rings, or aryl rings), having between 14 and 20 atoms, in which one or more carbon atoms are common to two adjacent rings. The rings in a polycyclic ring system may be fused (i.e., bicyclic or tricyclic), spirocyclic, or a combination thereof.

Polypeptide: The term “polypeptide”, as used herein, typically has its art-recognized meaning of a polymer of at least three amino acids or more. Those of ordinary skill in the art will appreciate that the term “polypeptide” is intended to be sufficiently general as to encompass not only polypeptides having a complete sequence recited herein, but also to encompass polypeptides that represent functional, biologically active, or characteristic fragments, portions or domains (e.g., fragments, portions, or domains retaining at least one activity) of such complete polypeptides. In some embodiments, polypeptides may contain L-amino acids, D-amino acids, or both and/or may contain any of a variety of amino acid modifications or analogs known in the art. Useful modifications include, e.g., terminal acetylation, amidation, methylation, etc. In some embodiments, polypeptides may comprise natural amino acids, non-natural amino acids, synthetic amino acids, and combinations thereof (e.g., may be or comprise peptidomimetics).

Polymer: As used herein, the term “polymer” refers to a composition comprising one or more molecules that comprise repeating units of one or more monomers. As used herein, “polymer” and “polymer composition” are used interchangeably, and unless otherwise specified, refer to a composition of polymer molecules. A person of skill in the art will appreciate that a polymer composition comprises polymer molecules having molecules of different lengths (e.g., comprising varying amounts of monomers). Polymer compositions described herein are characterized by one or more of a number average molecular weight (Mₙ), a weight average molecular weight (Mw), and/or a polydispersity index (PDI). Polymers described herein can also be characterized by the degree of polymerization (DP), which refers to the number of monomer units in the polymer. A polymer described herein can be a homopolymer, a heteropolymer, or a block-co-polymer. As used herein, a “homopolymer” refers to a polymer having a single type of monomer repeating throughout a polymer chain, e.g., -A-A-A-A-. As used herein, a “heteropolymer” refers to a polymer having more than one type (e.g., two or more) types of monomers present throughout a polymer chain, e.g., -A-B-A-B-A-. As used herein, a “block-co-polymer” refers to a polymer having an arrangement of blocks of polymerized monomers, e.g., -A-A-A-A-B-B-B-B- (a di block polymer) or -A-A-A-A-B-B-B-B-A-A-A- (a tri block polymer). Polymers described herein can be linear or branched.

Prevent or prevention: As used herein, the terms “prevent” or “prevention”, when used in connection with the occurrence of a disease, disorder, and/or condition, refer to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Ribonucleotide: As used herein, the term “ribonucleotide” encompasses unmodified ribonucleotides and modified ribonucleotides. For example, unmodified ribonucleotides include the purine bases adenine (A) and guanine (G), and the pyrimidine bases cytosine (C) and uracil (U). Modified ribonucleotides may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. The term “ribonucleotide” also encompasses ribonucleotide triphosphates including modified and non-modified ribonucleotide triphosphates.

Ribonucleic acid (RNA): As used herein, the term “RNA” refers to a polymer of ribonucleotides. In some embodiments, an RNA is single stranded. In some embodiments, an RNA is double stranded. In some embodiments, an RNA comprises both single and double stranded portions. In some embodiments, an RNA can comprise a backbone structure as described in the definition of “Nucleic acid/Polynucleotide” above. An RNA can be a regulatory RNA (e.g., siRNA, microRNA, etc.), or a messenger RNA (mRNA). In some embodiments where an RNA is a mRNA. In some embodiments where an RNA is a mRNA, a RNA typically comprises at its 3′ end a poly (A) region. In some embodiments where an RNA is a mRNA, an RNA typically comprises at its 5′ end an art-recognized cap structure, e.g., for recognizing and attachment of a mRNA to a ribosome to initiate translation. In some embodiments, a RNA is a synthetic RNA. Synthetic RNAs include RNAs that are synthesized in vitro (e.g., by enzymatic synthesis methods and/or by chemical synthesis methods). As used herein, “monomeric RNA”, also referred to as “monomolecular RNA”, refers to an individual RNA molecule that is not an aggregate, a dimer, trimer, or oligomer of RNA.

Sample: As used herein, the term “sample” typically refers to an aliquot of material obtained or derived from a source of interest. In some embodiments, a source of interest is a biological or environmental source. In some embodiments, a source of interest may be or comprise a cell, tissue, or organism, such as a microbe, a plant, or an animal (e.g., a human). In some embodiments, a source of interest is or comprises biological tissue or fluid. In some embodiments, a source of interest may be or comprise a preparation generated in a production run. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample.

Substituted or optionally substituted: As described herein, compounds of the invention may contain “optionally substituted” moieties. In general, the term “substituted,” whether preceded by the term “optionally” or not, means that one or more hydrogens of the designated moiety are replaced with a suitable substituent. “Substituted” applies to one or more hydrogens that are either explicit or implicit from the structure (e.g.,

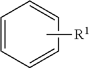

refers to at least

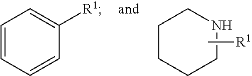

refers to at least

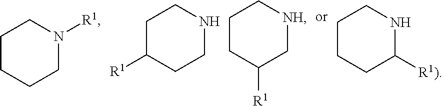

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes provided herein. Groups described as being “substituted” preferably have between 1 and 4 substituents, more preferably 1 or 2 substituents. Groups described as being “optionally substituted” may be unsubstituted or be “substituted” as described above.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —O₂; —CN; —N₃; (CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; N(R^{∘})C(S)NR^{∘}₂; —(CH₂)₀₋₄N(R^{∘})C(O)OR^{∘}; —N(R^{∘})N(R^{∘})C(O)R^{∘}; N(R^{∘})N(R^{∘})C(O)NR^{∘}₂; N(R^{∘})N(R^{∘})C(O)OR^{∘}; —(CH₂)₀₋₄C(O)R^{∘}; C(S)R^{∘}; —(CH₂)₀₋₄C(O)OR^{∘}; —(CH₂)₀₋₄C(O)SR^{∘}; (CH₂)₀₋₄C(O)OSiR^{∘}₃; —(CH₂)₀₋₄OC(O)R^{∘}; —OC(O)(CH₂)₀₋₄SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, (CH₂)₀₋₄OC(O)NR^{∘}₂; C(O)N(OR^{∘})R^{∘}; —C(O)C(O)R^{∘}; —C(O)CH₂C(O)R^{∘}; —C(NOR^{∘})R^{∘}; (CH₂)₀₋₄SSR^{∘}; —(CH₂)₀₋₄S(O)₂R^{∘}; —(CH₂)₀₋₄S(O)₂₀R^{∘}; —(CH₂)₀₋₄₀S(O)₂R^{∘}; —S(O)₂NR^{∘}₂; (CH₂)₀₋₄S(O)R^{∘}; N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; P(O)R^{∘}₂; OP(O)R^{∘}₂; —OP(O)(OR^{∘})₂; SiR^{∘}₃; —(C₁-4 straight or branched alkylene)O—N(R^{∘})₂; or —(C₁-4 straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R′, -(haloR^{●}), —(CH₂)₀₋₂OH, —(CH₂)₀₋₂OR^{●}, —(CH₂)₀₋₂CH(OR^{●})₂, O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR′, —(CH₂)₀₋₂SH, —(CH₂)₀₋₂NH₂, —(CH₂)₀₋₂NHR^{●}, —(CH₂)₀₋₂NR^{●}₂, —O₂, —SiR^{●}3, —OSiR^{●}₃, C(O)SR^{●}, —(C₁-4 straight or branched alkylene)C(O)OR^{●}₂, or —SSR^{●} wherein each R^{●} is unsubstituted or where preceded by “halo” is substituted only with one or more halogens, and is independently selected from C₁-4 aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O (“oxo”), ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁-6 aliphatic which may be substituted as defined below, or an unsubstituted 5- to 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁-6 aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, (haloR^{●}), OH, —OR^{●}, —O(haloR^{●}), —CN, —C(O)OH, —C(O)OR^{●}, —NH₂, —NHR^{●}, —NR^{∘}₂, or —O₂, wherein each R^{●} is unsubstituted or where preceded by “halo” is substituted only with one or more halogens, and is independently C₁-4 aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, S(O)₂R^{†}, S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3- to 6-membered saturated, partially unsaturated, or aryl ring having 0-4 heteroatoms independently selected from nitrogen, oxygen, or sulfur, or, notwithstanding the definition above, two independent occurrences of RT, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on the aliphatic group of RT are independently halogen, —R^{●}, (haloR^{●}), —OH, —OR^{●}, —O(haloR^{●}), —CN, —C(O)OH, —C(O)OR^{●}, —NH₂, —NHR^{●}, —NR^{∘}₂, or NO₂, wherein each R^{●} is unsubstituted or where preceded by “halo” is substituted only with one or more halogens, and is independently C₁-4 aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Small molecule: As used herein, the term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer.

Those of ordinary skill in the art, reading the present disclosure, will appreciate that certain small molecule compounds described herein, for example, may be provided and/or utilized in any of a variety of forms such as, for example, crystal forms (e.g., polymorphs, solvates, etc), salt forms, protected forms, pro-drug forms, ester forms, isomeric forms (e.g., optical and/or structural isomers), isotopic forms, etc.

Those of ordinary skill in the art will appreciate that certain small molecule compounds have structures that can exist in one or more steroisomeric forms. In some embodiments, such a small molecule may be utilized in accordance with the present disclosure in the form of an individual enantiomer, diastereomer or geometric isomer, or may be in the form of a mixture of stereoisomers; in some embodiments, such a small molecule may be utilized in accordance with the present disclosure in a racemic mixture form.

Those of skill in the art will appreciate that certain small molecule compounds have structures that can exist in one or more tautomeric forms. In some embodiments, such a small molecule may be utilized in accordance with the present disclosure in the form of an individual tautomer, or in a form that interconverts between tautomeric forms.

Those of skill in the art will appreciate that certain small molecule compounds have structures that permit isotopic substitution (e.g., ²H or ³H for H; ¹¹C, ¹³C or ¹⁴C for ¹²C; ¹³N or ¹⁵N for ¹⁴N; ¹⁷O or ¹⁸O for ¹⁶O; ³⁶Cl for ³⁵Cl or ³⁷Cl; ¹⁸F for ¹⁹F; ¹³¹I for ¹²⁷I; etc.). In some embodiments, such a small molecule may be utilized in accordance with the present disclosure in one or more isotopically modified forms, or mixtures thereof.

In some embodiments, reference to a particular small molecule compound may relate to a specific form of that compound. In some embodiments, a particular small molecule compound may be provided and/or utilized in a salt form (e.g., in an acid-addition or base-addition salt form, depending on the compound); in some such embodiments, the salt form may be a pharmaceutically acceptable salt form.

In some embodiments, where a small molecule compound is one that exists or is found in nature, that compound may be provided and/or utilized in accordance in the present disclosure in a form different from that in which it exists or is found in nature. Those of ordinary skill in the art will appreciate that, in some embodiments, a preparation of a particular small molecule compound that contains an absolute or relative amount of the compound, or of a particular form thereof, that is different from the absolute or relative (with respect to another component of the preparation including, for example, another form of the compound) amount of the compound or form that is present in a reference preparation of interest (e.g., in a primary sample from a source of interest such as a biological or environmental source) is distinct from the compound as it exists in the reference preparation or source. Thus, in some embodiments, for example, a preparation of a single stereoisomer of a small molecule compound may be considered to be a different form of the compound than a racemic mixture of the compound; a particular salt of a small molecule compound may be considered to be a different form from another salt form of the compound; a preparation that contains only a form of the compound that contains one conformational isomer ((Z) or (E)) of a double bond may be considered to be a different form of the compound from one that contains the other conformational isomer ((E) or (Z)) of the double bond; a preparation in which one or more atoms is a different isotope than is present in a reference preparation may be considered to be a different form; etc.

Those skilled in the art will further appreciate that, in small molecule structures, the symbol [symbol] , as used herein, refers to a point of attachment between two atoms.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Treat: As used herein, the terms “treat,” “treatment,” or “treating” refer to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example, for the purpose of decreasing the risk of developing pathology associated with the disease, disorder, and/or condition.

### Complexes

The present disclosure provides, among other things, complexes for delivery of a nucleic acid, e.g., RNA. In some embodiments, the present disclosure provides a complex comprising a first cationic polymer, a second cationic polymer, and a monomeric RNA molecule, wherein the first cationic polymer comprises greater than 250 repeating monomer units, and the second cationic polymer comprises 200 repeating monomer units, or less. In some embodiments, a complex described herein comprises a monomeric RNA molecule.

As described generally herein, a complex comprises a first cationic polymer, a second cationic polymer, and RNA (e.g., monomeric RNA, as described herein). Examples of cationic polymers (e.g., a first cationic polymer and a second cationic polymer) and conditions suitable for forming complexes are described in PCT App. Pub. No. WO 2021/001417, which is incorporated herein by reference in its entirety. Such complexes are characterized by an “N/P ratio,” which is the molar ratio of total cationic (e.g., nitrogen) groups (the “N” in N/P) in the cationic polymers to the anionic (phosphate) groups (the “P” in N/P) in RNA. It is understood that a cationic group is one that is either in cationic form (e.g., N⁺), or one that is ionizable to become cationic. Use of a single number in an N/P ratio (e.g., an N/P ratio of about 5) is intended to refer to that number over 1, e.g., an N/P ratio of about 5 is intended to mean a ratio of about 5:1.

In some embodiments, an N/P ratio for a complex described herein is greater than or equal to about 5. In some embodiments, an N/P ratio for a complex described herein is greater than or equal to about 10. In some embodiments, an N/P ratio for a complex described herein is greater than or equal to about 12. In some embodiments, an N/P ratio for a complex described herein is greater than or equal to about 20. In some embodiments, an N/P ratio for a complex described herein is greater than or equal to about 40. In some embodiments, an N/P ratio for a complex described herein is greater than or equal to about 70. In some embodiments, an N/P ratio for a complex described herein is greater than or equal to about 100. In some embodiments, an N/P ratio for a complex described herein is greater than or equal to about 200. In some embodiments, an N/P ratio for a complex described herein is greater than or equal to about 300. In some embodiments, an N/P ratio for a complex described herein is from about 5 to about 20 (e.g., about 5, about 6, about 7, about 8, about 9, about 10, about 11, about 12, about 13, about 14, about 15, about 16, about 17, about 18, about 19, or about 20). In some embodiments, an N/P ratio for a complex described herein is from about 10 to about 50. In some embodiments, an N/P ratio for a complex described herein is from about 10 to about 70. In some embodiments, an N/P ratio for a complex described herein is from about 10 to about 120. In some embodiments, an N/P ratio for a complex described herein is from about 40 to about 70. In some embodiments, an N/P ratio for a complex described herein is from about 40 to about 120 (e.g., about 40, about 50, about 60, about 70, about 80, about 90, about 100, about 110, or about 120). In some embodiments, an N/P ratio for a complex described herein is from about 70 to about 120. In some embodiments, an N/P ration is greater than or equal to 48. In some embodiments, an N/P ratio is from about 48 to about 300. In some embodiments, an N/P ratio is from about 60 to about 200. In some embodiments, an N/P ratio is from about 80 to about 150.

In some embodiments, a complex described herein is characterized by an RNA concentration. In some embodiments, an RNA concentration in a complex is greater than or equal to about 0.05 mg/mL. In some embodiments, an RNA concentration in a complex is greater than or equal to about 0.1 mg/mL. In some embodiments, an RNA concentration in a complex is greater than or equal to about 0.25 mg/mL. In some embodiments, an RNA concentration in a complex is greater than or equal to about 0.5 mg/mL. In some embodiments, an RNA concentration in a complex is from about 0.01 mg/mL to about 0.5 mg/mL. In some embodiments, an RNA concentration in a complex is from about 0.1 mg/mL to about 0.25 mg/mL. In some embodiments, an RNA concentration in a complex is about 0.1 mg/mL, about 0.15 mg/mL, about 0.2 mg/mL, about 0.25 mg/mL, about 0.3 mg/mL, about 0.35 mg/mL, about 0.4 mg/mL, about 0.45 mg/mL, or about 0.5 mg/mL. In some embodiments, an RNA concentration in a complex is about 0.1 mg/mL.

In some embodiments, an RNA concentration in a complex is about 0.1 mg/mL, and an N/P ratio is greater than or equal to about 45. In some embodiments, an RNA concentration in a complex is about 0.1 mg/mL, and an N/P ratio is from about 48 to about 300. In some embodiments, an RNA concentration in a complex is about 0.1 mg/mL, and an N/P ratio is from about 60 to about 200. In some embodiments, an RNA concentration in a complex is about 0.1 mg/mL, and an N/P ratio is from about 80 to about 150.

In some embodiments, an RNA concentration in a complex is about 0.05 mg/mL, and an N/P ratio is greater than or equal to about 5. In some embodiments, an RNA concentration in a complex is about 0.05 mg/mL, and an N/P ratio is greater than or equal to about 12. In some embodiments, an RNA concentration in a complex is about 0.05 mg/mL, and an N/P ratio is greater than or equal to about 45. In some embodiments, an RNA concentration in a complex is about 0.05 mg/mL, and an N/P ratio is greater than about 60.

In some embodiments, a complex described herein comprises monomeric RNA. “Monomeric RNA,” also referred to as “monomolecular RNA”, as used herein, refers to RNA that is an individual RNA molecule, and not an aggregate, or as a dimer, trimer, or oligomer of RNA. Therefore, reference to a complex comprising monomeric RNA is understood to be a complex that with a single RNA molecule that is not an aggregate, or is a dimer, trimer, or oligomer of RNA.

In some embodiments, a complex described herein is characterized by a diameter of from about 10 nm to about 70 nm. In some embodiments, a complex described herein is characterized by a diameter of from about 20 nm to about 50 nm. In some embodiments, a complex described herein is characterized by a diameter of about 20 nm, about 25 nm, about 30 nm, about 35 nm, about 40 nm, about 45 nm, or about 50 nm. In some embodiments, a complex described herein is characterized by a diameter of less than about 50 nm. Methods of characterizing a diameter for a complex are described in PCT App. Pub. No. WO 2021/001417

It is understood that complexes described herein are included as part of a solution or formulation. As such, many complexes will make up a solution or formulation. Characterization of any complex within is intended to represent either an average of all complexes within a given solution or formulation, as well as individual complexes when characterized according to methods known in the art.

### Cationic Polymers

As described generally above, a complex described herein comprises a first cationic polymer having greater than 250 repeating monomer units, and a second cationic polymer having 200 repeating monomer units, or less. In some embodiments, a first or second cationic polymer is a polycationic polymer, e.g., a polymer having one or more cationic or ionizable groups. As described herein, a “cationic” group is a group having a net positive charge. As used herein, an “ionizable” group is a group that may have a neutral charge at a certain pH, but may become charged (e.g., cationic) at a different pH. For example, in some embodiments, an ionizable group becomes cationic (i.e., positively charged) at physiological pH (e.g., a pH of about 7.4). Accordingly, it is understood that reference to a cationic polymer refers to both the neutral form and the charged (i.e., ionic) form. A person of skill in the art will also appreciate that cationic groups described herein can also exist as a salt (e.g., a pharmaceutically acceptable salt) that comprises a cationic group and one or more suitable counterions. For example, in some embodiments, a cationic group described herein comprises ammonium chloride. Suitable counterions include halogens (e.g., Br⁻, Cl⁻, I⁻, F⁻), acetates (e.g., C(O)O⁻), and the like. For additional examples, see the definition for pharmaceutically acceptable salts described herein.

In some embodiments, one or more cationic or ionizable groups comprise a nitrogen atom. Cationic polymers useful for preparing complexes described herein can be homopolymers heteropolymers, or block-co-polymers.

In some embodiments, a first cationic polymer is or comprises poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof.

In some embodiments, a first cationic polymer is a homopolymer. In some embodiments, a first cationic polymer is a homopolymer selected from poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof.

It is understood that a first cationic polymer described herein can be linear or branched. In some embodiments, a first cationic polymer is linear. In some embodiments, a first cationic polymer is a linear polymer selected from poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof. In some embodiments, a first cationic polymer is a branched polymer selected from poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof. In some embodiments, a first cationic polymer is linear poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof.

In some embodiments, a first cationic polymer is poly(ethylenimine). In some embodiments, a first cationic polymer is poly(propylenimine). In some embodiments, a first cationic polymer is polybrene. In some embodiments, a first cationic polymer is polyallylamine. In some embodiments, a first cationic polymer is polyvinylamine. In some embodiments, a first cationic polymer is polyamidoamine. In some embodiments, a first cationic polymer is poly-L-lysine. In some embodiments, a first cationic polymer is poly-L-arginine. In some embodiments, a first cationic polymer is poly-L-histidine. In some embodiments, a first cationic polymer is poly(2-aminoethyl methacrylate).

In some embodiments, a first cationic polymer is a heteropolymer comprising copolymers of one or more of poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof. In some embodiments, a first cationic polymer is a heteropolymer comprising poly(ethylenimine) and poly(propylenimine). In some embodiments, a first cationic polymer is a linear heteropolymer comprising copolymers of one or more of poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof. In some embodiments, a first cationic polymer is a heteropolymer comprising poly(ethylenimine) and poly(propylenimine).

In some embodiments, a first cationic polymer is a block-co-polymer comprising blocks of polymers selected from poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof. In some embodiments, a first cationic polymer is a block-co-polymer of poly(ethylenimine) and poly(propylenimine) (i.e., poly(ethylenimine)-block-poly(propylenimine)) In some embodiments, a first cationic polymer is a linear block-co-polymer comprising blocks of polymers selected from poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate). In some embodiments, a first cationic polymer is a linear block-co-polymer of poly(ethylenimine) and poly(propylenimine) (i.e., poly(ethylenimine)-block-poly(propylenimine)).

In some embodiments, a first cationic polymer has between 250 and 2000 repeating monomer units. In some embodiments, a first cationic polymer has between 500 and 2000 repeating monomer units. In some embodiments, a first cationic polymer has between 1000 and 2000 repeating monomer units. In some embodiments, a first cationic polymer has between 1500 and 2000 repeating monomer units. In some embodiments, a first cationic polymer comprises about 250, about 300, about 350, about 400, about 500, about 600, about 700, about 800, about 900, about 1000, about 1100, about 1200, about 1300, about 1400, about 1500, about 1600, about 1700, about 1800, about 1900, or about 2000 monomer units.

In some embodiments, a first cationic polymer is a polymer or copolymer comprising blocks of formula I and/or II:

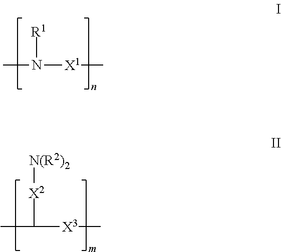

or a pharmaceutically acceptable salt thereof, wherein:
R¹ is H, —C(O)-optionally substituted C₁-C₆ aliphatic, optionally substituted C₁-C₆ aliphatic or G;
each R² is independently H, optionally substituted C₁-C₆ aliphatic, or G;
X¹ and X³ are each independently C₁-C₆ aliphatic;
X² is a bond or optionally substituted C₁-C₆ aliphatic;
m is an integer between 2 and 2000;
n is an integer between 2 and 2000;
each G is independently:

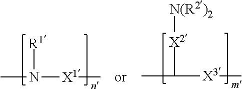

R^{1′} is H, —C(O)-optionally substituted C₁-C₆ aliphatic, optionally substituted C₁-C₆ aliphatic, or G′;
each R^{2′} is independently H, optionally substituted C₁-C₆ aliphatic, or G′;
X^{1′} and X^{3′} are each independently optionally substituted C₁-C₆ aliphatic;
X^{2′} is a bond or optionally substituted C₁-C₆ aliphatic;
n′ is an integer between 2 and 2000;
m′ is an integer between 2 and 2000;
each G′ is independently:

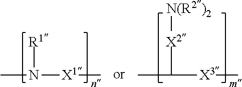

R^{1″} is H, —C(O)-optionally substituted C₁-C₆ aliphatic, or optionally substituted C₁-C₆ aliphatic; each R^{2″} is independently H or C₁-C₆ aliphatic;
X^{1″} and X^{3″} are each independently optionally substituted C₁-C₆ aliphatic;
X^{2″} is a bond or optionally substituted C₁-C₆ aliphatic;
n″ is an integer between 2 and 2000; and
m″ is an integer between 2 and 2000.

In some embodiments, a first cationic polymer is a polymer described herein, having a number average molecular weight (Mₙ) of about 600 Daltons (Da) to about 400,000 Da. In some embodiments, a first cationic polymer has a Mₙ of about 1,000 Da to about 300,000 Da. In some embodiments, a first cationic polymer has a Mₙ of about 10,000 to about 250,000 Da. In some embodiments, a first cationic polymer has a Mₙ of about 10,000 to about 120,000 Da. In some embodiments, a first cationic polymer has a Mₙ of about 20,000 to about 120,000 Da.

In some embodiments, a second cationic polymer is or comprises a polypeptide, a peptide, a protein, a polysaccharide, or a polyimine.

In some embodiments, a second cationic polymer is or comprises a polypeptide or a peptide. In some embodiments, a second cationic polymer is a polypeptide. In some embodiments, a second cationic polymer is a polypeptide comprising monomers of natural amino acids (a canonical amino acid), or a non-natural amino acid (i.e., a non-canonical amino acid). In some embodiments, a non-natural amino acid is a compound of formula III:

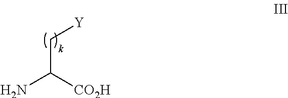

or a pharmaceutically acceptable salt thereof, wherein Y is —R³, —SR³, —N(R³)₂, —N³, —OR³, or

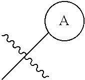

A is optionally substituted C₃-C₁₂ cycloaliphatic, optionally substituted C₆-C₁₂ aryl, optionally substituted 4- to 12-membered heterocycle comprising 1 to 4 heteroatoms selected from N, O, and S, or optionally substituted 5- to 12-membered heteroaryl comprising 1 to 4 heteroatoms selected from N, O, and S;
each R³ is independently selected from H, optionally substituted C₁-C₁₂ aliphatic, and optionally substituted 3- to 12-membered heteroaliphatic; and
k is 0-6.

As described herein, reference to a natural amino acid refers to “canonical” amino acids, which are those amino acids that are capable of being encoded directly by the codons of the universal genetic code. These amino acids include alanine, arginine, asparagine, aspartate, cysteine, glutamine, glycine, histidine, isoleucine, leucine, lysine, methionine, phenylalanine, proline, serine, threonine, tryptophan, tyrosine, and valine.

In some embodiments, a second cationic polymer is a polypeptide having 25% or more monomers selected from arginine, histidine, lysine, and a non-natural amino acid. In some embodiments, a second cationic polymer is a polypeptide having 25% or more monomers selected from arginine, histidine, lysine, and a non-natural amino acid of formula III.

In some embodiments, a polypeptide is a TAT peptide, a TAT-derived peptide, poly-L-arginine, or a poly-L-arginine-derived peptide. As used herein, a TAT peptide is derived from the transactivator of transcription (TAT) of human immunodeficiency virus type 1 and is a cell-penetrating peptide. As described herein, a TAT peptide comprises the following amino acid sequence GRKKRRQRRRPQ (Gly-Arg-Lys-Lys-Arg-Arg-Gln-Arg-Arg-Arg-Pro-Gln) (SEQ ID NO: 1). A TAT-derived peptide is a TAT peptide wherein one or more amino acids of the TAT peptide is modified or otherwise replaced, for example with a non-natural amino acid of Formula III, or additional amino acids or non-natural amino acids of Formula III are added to the TAT peptide. For example, in some embodiments, a TAT-derived peptide is a polypeptide of the sequence CHHHHHRKKRRQRRRRHHHHHC (Cys-His-His-His-His-His-Arg-Lys-Lys-Arg-Arg-Gln-Arg-Arg-Arg-Arg-His-His-His-His-His-Cys) (SEQ ID NO:2).

In some embodiments, a polypeptide is poly-L-arginine. In some embodiments, a polypeptide is a poly-L-arginine-derived peptide. As used herein, a poly-L-arginine derived peptide is poly-L-arginine, wherein one or more arginine monomers are replaced with another amino acid (e.g., a natural or non-natural amino acid, such as a non-natural amino acid of formula III), or an arginine monomer is chemically modified according to methods known to those of skill in the art.

In some embodiments, a second cationic polymer is a polysaccharide. In some embodiments, a polysaccharide is chitosan or diethylaminoethyl dextran.

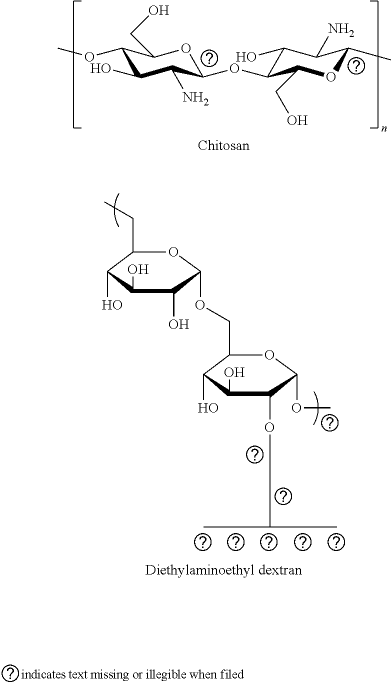

In some embodiments, a second cationic polymer is a polyimine. In some embodiments, a polyimine is poly(ethylenimine), poly(propylenimine), poly(amidoamine), polyaminoester, poly(allylamine), polyvinylamine, poly(dimethylaminoethyl methacrylate), or polybrene.

In some embodiments, a second cationic polymer is poly(ethylenimine). In some embodiments, a second cationic polymer is poly(propylenimine). In some embodiments, a second cationic polymer is poly(amidoamine). In some embodiments, a second cationic polymer is polyaminoester. In some embodiments, a second cationic polymer is poly(allylamine). In some embodiments, a second cationic polymer is polyvinylamine. In some embodiments, a second cationic polymer is poly(dimethylaminoethyl methacrylate). In some embodiments, a second cationic polymer is polybrene (otherwise referred to as hexadimethrine bromide).

In some embodiments, a second cationic polymer comprises less than 200 monomer units. In some embodiments, a second cationic polymer comprises less than 100 monomer units. In some embodiments, a second cationic polymer comprises about 30 monomer units or less. In some embodiments, a second cationic polymer comprises about 20 monomer units. In some embodiments, a second cationic polymer comprises about 10, about 20, about 30, about 40, about 50, about 60, about 70, about 80, about 90, about 100, about 120, about 130, about 140, about 160, about 180, or about 200 monomer units.

In some embodiments, a molar ratio of a first cationic polymer to a second polymer in a complex described herein is from about 50:50 to about 5:95. In some embodiments, a molar ratio of a first cationic polymer to a second polymer in a complex described herein is from about 40:50 to about 10:90. In some embodiments, a molar ratio of a first cationic polymer to a second polymer in a complex described herein is from about 25:75 to about 12.5:87.5. In some embodiments, a molar ratio of a first cationic polymer to a second polymer in a complex described herein is about 50:50, about 45:55, about 40:50, about 35:65, about 30:70, about 25:75, about 20:80, about 19:81, about 18:82, about 17:83, about 16:84, about 15:85, about 14:86, about 13:87, about 12.5:87.5, about 12:88, about 11.5:88.5, about 11:89, about 10.5:89.5, about 10:90, about 9.5:90.5, about 9:91, about 8.5:91.5, about 8:92, about 7.5:92.5, about 7:93, about 6.5:93.5, about 6:94, about 5.5:94.5, or about 5:95.

In some embodiments, provided compounds are provided and/or utilized in a salt form (e.g., a pharmaceutically acceptable salt form). Reference to a compound provided herein is understood to include reference to salts thereof, unless otherwise indicated.

### RNA

In some embodiments, a complex described herein comprises a first cationic polymer, a second cationic polymer, and a monomeric RNA molecule.

In some embodiments, an RNA amenable to technologies described herein is a single-stranded RNA. In some embodiments, an RNA as disclosed herein is a linear RNA. In some embodiments, a single-stranded RNA is a non-coding RNA in that its nucleotide sequence does not include an open reading frame (or complement thereof). In some embodiments, a single-stranded RNA has a nucleotide sequence that encodes (or is the complement of a sequence that encodes) a polypeptide or a plurality of polypeptides (e.g., epitopes) of the present disclosure.

In some embodiments, an RNA is or comprises an siRNA, an miRNA, or other non-coding RNA.

In many embodiments, a relevant RNA includes at least one open reading frame (ORF) (e.g., is an mRNA); in some embodiments, a relevant RNA includes a single ORF; in some embodiments, a relevant RNA includes more than one ORF.

In some embodiments, an RNA comprises an ORF, e.g., encoding a polypeptide of interest or encoding a plurality of polypeptides of interest. In some embodiments, an RNA produced in accordance with technologies provided herein comprises a plurality of ORFs (e.g., encoding a plurality of polypeptides). In some embodiments, an RNA produced in accordance with technologies herein comprises a single ORF that encodes a plurality of polypeptides. In some such embodiments, polypeptides are or comprise antigens or epitopes thereof (e.g., relevant antigens).

In some embodiments, an ORF for use in accordance with the present disclosure encodes a polypeptide that includes a signal sequence, e.g., that is functional in mammalian cells, such as an intrinsic signal sequence or a heterologous signal sequence. In some embodiments, a signal sequence directs secretion of an encoded polypeptide, in some embodiments, a signal sequence directs transport of an encoded polypeptide into a defined cellular compartment, preferably the cell surface, the endoplasmic reticulum (ER) or the endosomal-lysosomal compartment.

In some embodiments, an ORF encodes a polypeptide that includes a multimerization element (e.g., an intrinsic or heterologous multimerization element). In some embodiments, an ORF that encodes a surface polypeptide (e.g., that includes a signal sequence directing surface localization) includes a multimerization element.

In some embodiments, an ORF encodes a polypeptide that includes a transmembrane element or domain.

In some embodiments, an ORF is codon-optimized for expression in a cells of a particular host, e.g., a mammalian host, e.g., a human.

In some embodiments, an RNA includes unmodified uridine residues; an RNA that includes only unmodified uridine residues may be referred to as a “uRNA”. In some embodiments, an RNA includes one or more modified uridine residues; in some embodiments, such an RNA (e.g., an RNA including entirely modified uridine residues) is referred to as a “modRNA”. In some embodiments, an RNA may be a self-amplifying RNA (saRNA). In some embodiments, an RNA may be a trans-amplifying RNA (see, for example, WO2017/162461).

In some embodiments, a relevant RNA includes a polypeptide-encoding portion or a plurality of polypeptide-encoding portions. In some particular embodiments, such a portion or portions may encode a polypeptide or polypeptides that is or comprises a biologically active polypeptide or portion thereof (e.g., an enzyme or cytokine or therapeutic protein such as a replacement protein or antibody or portion thereof). In some particular embodiments, such a portion or portions may encode a polypeptide or polypeptides that is or comprises an antigen (or an epitope thereof), a cytokine, an enzyme, etc. In some embodiments, an encoded polypeptide or polypeptides may be or include one or more neoantigens or neoepitopes associated with a tumor. In some embodiments, an encoded polypeptide or polypeptides may be or include one or more antigens (or epitopes thereof) of an infectious agent (e.g., a bacterium, fungus, virus, etc.). In certain embodiments, an encoded polypeptide may be a variant of a wild type polypeptide.

In some embodiments, a single-stranded RNA (e.g., mRNA) may comprise a secretion signal-encoding region (e.g., a secretion signal-encoding region that allows an encoded target entity or entities to be secreted upon translation by cells). In some embodiments, such a secretion signal-encoding region may be or comprise a non-human secretion signal. In some embodiments, such a secretion signal-encoding region may be or comprise a human secretion signal.

In some embodiments, a single-stranded RNA (e.g., mRNA) may comprise at least one non-coding element (e.g., to enhance RNA stability and/or translation efficiency). Examples of non-coding elements include but are not limited to a 3′ untranslated region (UTR), a 5′ UTR, a cap structure (e.g., in some embodiments, an enzymatically-added cap; in some embodiments, a co-transcriptional cap), a poly adenine (polyA) tail (e.g., that, in some embodiments, may be or comprise 100 A residues or more, and/or in some embodiments may include one or more “interrupting” [i.e., non-A] sequence elements), and any combinations thereof. Exemplary embodiments of such non-coding elements may be found, for example, in WO2011015347, WO2017053297, U.S. Pat. Nos. 10,519,189, 10,494,399, WO2007024708, WO2007036366, WO2017060314, WO2016005324, WO2005038030, WO2017036889, WO2017162266, and WO2017162461, each of which is incorporated herein by referenced in its entirety.

### Formats

At least four formats useful for RNA pharmaceutical compositions (e.g., immunogenic compositions or vaccines) have been developed, namely non-modified uridine containing mRNA (uRNA), nucleoside-modified mRNA (modRNA), self-amplifying mRNA (saRNA), and trans-amplifying RNAs.

Features of a non-modified uridine platform may include, for example, one or more of intrinsic adjuvant effect, good tolerability and safety, and strong antibody and T cell responses.

Features of modified uridine (e.g., pseudouridine) platform may include reduced adjuvant effect, blunted immune innate immune sensor activating capacity and thus augmented antigen expression, good tolerability and safety, and strong antibody and CD4-T cell responses. As noted herein, the present disclosure provides an insight that such strong antibody and CD4 T cell responses may be particularly useful for vaccination.

Features of self-amplifying platform may include, for example, long duration of polypeptide (e.g., protein) expression, good tolerability and safety, higher likelihood for efficacy with very low vaccine dose.

In some embodiments, a self-amplifying platform (e.g., RNA) comprises two nucleic acid molecules, wherein one nucleic acid molecule encodes a replicase (e.g., a viral replicase) and the other nucleic acid molecule is capable of being replicated (e.g., a replicon) by said replicase in trans (trans-replication system). In some embodiments, a self-amplifying platform (e.g., RNA) comprises a plurality of nucleic acid molecules, wherein said nucleic acids encode a plurality of replicases and/or replicons.

In some embodiments, a trans-replication system comprises the presence of both nucleic acid molecules in a single host cell.

In some such embodiments, a nucleic acid encoding a replicase (e.g., a viral replicase) is not capable of self-replication in a target cell and/or target organism. In some such embodiments, a nucleic acid encoding a replicase (e.g., a viral replicase) lacks at least one conserved sequence element important for (−) strand synthesis based on a (+) strand template and/or for (+) strand synthesis based on a (−) strand template.

In some embodiments, a self-amplifying RNA comprises a 5′-cap; in some trans-replication systems, at least an RNA encoding a replicase is capped. Without wishing to be bound by any one theory, it has been found that a 5′-cap can be important for high level expression of a gene of interest in trans.

In some embodiments, a self-amplifying platform does not require propagation of virus particles (e.g., is not associated with undesired virus-particle formation). In some embodiments, a self-amplifying platform is not capable of forming virus particles.

In some embodiments, an RNA may comprise an Internal Ribosomal Entry Site (IRES) element. In some embodiments, an RNA does not comprise an IRES site; in particular, in some embodiments, an saRNA does not comprise an IRES site. In some such embodiments, translation of a gene of interest and/or replicase is not driven by an IRES element. In some embodiments, an IRES element is substituted by a 5′-cap. In some such embodiments, substitution by a 5′-cap does not affect the sequence of a polypeptide encoded by an RNA.

In some embodiments, a complex described herein comprises mRNA, modRNA or saRNA. In some embodiments, a complex comprises mRNA. In some embodiments, a complex comprises modRNA. In some embodiments, a complex comprises saRNA.

### Methods of Delivery

The present disclosure provides, among other things, a complex (e.g., a pharmaceutical composition or a pharmaceutical formulation, as referred to herein) to be administered to a subject. For example, in some embodiments, a complex is administered as a monotherapy. In some embodiments, a complex is administered as part of a combination therapy. In some embodiments, a concentration of RNA in a pharmaceutical composition described herein is of about 0.01 mg/mL to about 0.5 mg/mL, or about 0.05 mg/mL to about 0.1 mg/mL.

Pharmaceutical compositions may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated herein by reference in its entirety) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, an excipient is approved for use in humans and for veterinary use. In some embodiments, an excipient is approved by the United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

In some embodiments, pharmaceutical formulations described herein can further comprise a buffering agent. In some embodiments, a buffering agent is selected from 4-(2-hydroxyethyl)-1-piperazineethanesulfonic acid (HEPES), 3-morpholino-2-hydroxypropanesulfonic acid (MOPSO), 2-(N-morpholino) ethanesulfonic acid (MES), Bis-tris buffering systems, carboxylic acid buffering systems, phosphatic acid buffering systems, or citric acid buffering systems.

General considerations in the formulation and/or manufacture of pharmaceutical agents may be found, for example, in Remington: The Science and Practice of Pharmacy 21st ed., Lippincott Williams & Wilkins, 2005 (incorporated herein by reference in its entirety).

In some embodiments, pharmaceutical compositions provided herein may be formulated with one or more pharmaceutically acceptable carriers or diluents as well as any other known adjuvants and excipients in accordance with conventional techniques such as those disclosed in Remington: The Science and Practice of Pharmacy 21st ed., Lippincott Williams & Wilkins, 2005 (incorporated herein by reference in its entirety).

Pharmaceutical complexes and compositions described herein can be administered by appropriate methods known in the art. As will be appreciated by a skilled artisan, the route and/or mode of administration may depend on a number of factors, including, e.g., but not limited to stability and/or pharmacokinetics and/or pharmacodynamics of pharmaceutical compositions described herein.

In some embodiments, pharmaceutical compositions described herein are formulated for parenteral administration, which includes modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

In some embodiments, pharmaceutical compositions described herein are formulated for intravenous administration. In some embodiments, pharmaceutically acceptable carriers that may be useful for intravenous administration include sterile aqueous solutions or dispersions and sterile powders for preparation of sterile injectable solutions or dispersions.

In some particular embodiments, pharmaceutical compositions described herein are formulated for subcutaneous (s.c.) administration. In some particular embodiments, pharmaceutical compositions described herein are formulated for intramuscular (i.m.) administration. In some embodiments, pharmaceutical compositions described herein are formulation for intranasal administration. In some embodiments, pharmaceutical compositions described herein are formulation for intravenous (i.v.) administration.

Therapeutic compositions typically must be sterile and stable under the conditions of manufacture and storage. The composition can be formulated as a solution, dispersion, powder (e.g., lyophilized powder), microemulsion, lipid nanoparticles, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. In some embodiments, prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration.

In some embodiments, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Examples of suitable aqueous and nonaqueous carriers which may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the presence of microorganisms may be ensured both by sterilization procedures, and by the inclusion of various antibacterial and antifungal agents, for example, paraben, chlorobutanol, phenol sorbic acid, and the like. It may also be desirable to include isotonic agents, such as sugars, sodium chloride, and the like into pharmaceutical compositions described herein. In addition, prolonged absorption of the injectable pharmaceutical form may be brought about by the inclusion of agents which delay absorption such as aluminum monostearate and gelatin.

Formulations of pharmaceutical compositions described herein may be prepared by any method known or hereafter developed in the art of pharmacology. In general, such preparatory methods include the step of bringing active ingredient(s) into association with a diluent or another excipient and/or one or more other accessory ingredients, and then, if necessary and/or desirable, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of at least one RNA product produced using a system and/or method described herein.

In some embodiments, an active agent that may be included in a pharmaceutical composition described herein is or comprises a therapeutic agent administered in a combination therapy described herein. Pharmaceutical compositions described herein can be administered in combination therapy, i.e., combined with other agents. In some embodiments, such therapeutic agents may include agents leading to depletion or functional inactivation of regulatory T cells. For example, in some embodiments, a combination therapy can include a provided pharmaceutical composition with at least one immune checkpoint inhibitor.

In some embodiments, pharmaceutical composition described herein may be administered in conjunction with radiotherapy and/or autologous peripheral stem cell or bone marrow transplantation.

In some embodiments, a pharmaceutical composition described herein can be frozen to allow long-term storage.

Although the descriptions of pharmaceutical compositions provided herein are principally directed to pharmaceutical compositions that are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation.

It is also appreciated that different methods of administration can be used to treat diseases, disorders, or conditions described herein. For example, in some embodiments, a composition comprising a complex described herein is administered a method described herein in one or more doses (e.g., one or more doses by parenteral means, intranasal means, oral, and the like). In some embodiments, a first dose is administered by a first means, and a second dose is administered by the same or different means. For example, in some embodiments, a first dose of a composition comprising a complex described herein is administered by intramuscular, subcutaneous, or intravenous means, and a second dose of a composition comprising a complex described herein is administered by intranasal means. In some embodiments, doses can be administered in a routine manner by any means described herein after an initial dose. For example, in some embodiments, a first dose is administered by intramuscular, subcutaneous, or intravenous means, and subsequent doses are administered by intranasal means.

In some embodiments, a first dose is administered, and a second dose is administered at a period of time after the first dose. In some embodiments, a period of time between a first and second dose is between one day and one year. In some embodiments, a period of time between a first dose and a second dose is between one week and six months. In some embodiments, a period of time between a first dose and a second dose is between one month and three months.

In some embodiments, subsequent doses after a first dose are administered in regular intervals. For example, in some embodiments, a subsequent dose is administered once per year after administration of a first dose.

### Methods of Use

Complexes described herein are useful in the treatment and prophylaxis in a subject of diseases, disorders, and conditions described herein. In some embodiments, a disease, disorder, or condition is an infectious disease, cancer, an autoimmune disease, or a rare disease.

In some embodiments, an infectious disease is caused by or associated with a viral pathogen. In some embodiments, a viral pathogen is of a family selected from poxviridae, rhabdoviridae, filoviridae, paramyxoviridae, hepadnaviridae, coronaviridae, caliciviridae, picornaviridae, reoviridae, retroviridae, and orthomyxoviridae. In some embodiments, an infectious disease is a virus selected from SARS-COV-2, influenza, Crimean-Congo Hemorrhagic Fever (CCHF), Ebola virus, Lassa virus, Marburg virus, HIV, Nipah virus, and MERS-COV.

In some embodiments, an infectious disease is caused by or associated with a bacterial pathogen. In some embodiments, a bacterial pathogen is of a species selected from Actinomyces israelii, bacillus antracis, Bacteroides fragilis, Bordetella pertussis, Borrelia burgdorferi, Borrelia garinii, Borrelia afzelii, Borrelia recurrentis, Brucella abortus, Brucella canis, Brucella melitensis, Brucella suis, Campolobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium idphteriae, Ehrlichia canis, Ehrlichia chaffeensis, Enterococcus faecalis, Enterococcus faecium, Escherichia coli, Francisella tularensis, Haemophilus influenzae, Helicobacter pylori, Klebsiella pneumoniae, Legionella pneumophila, Leptospira, Listeria monocytogenes, Mycobacterium leprae, Mycobacterium tuberculosis, Mycoplasma pneumoniae, Neisseria gonorrhoeae, Neisseria meningitidis, Pseudomonas aeruginosa, Nocardia asteroids, Rickettsia ricektssii, Salmonella typhi, Salmonella typhimurium, Shigella sonnei, Shigella dysenteriae, Staphylococcus aureus, Staphylococcus epidermidis, Staphylococcus saprophyticus, Streptococcus agalactiae, Streptococcus pneumoniae, Streptococcus pyogenes, Streptococcus viridans, Treponema pallidum, Vibrio cholerae, and Yersinia pestis.

In some embodiments, an infectious disease is caused by or associated with a parasite. In some embodiments, a parasite is of a family selected from Plasmodium, Leishmania, Cryptosporidium, Entamoeba, Trypanosomas, Schistosomes, Ascaris, Echinococcus and Taeniidae.

In some embodiments, a disease, disorder, or condition is a cancer. In some embodiments, a cancer is selected from bladder cancer, breast cancer, colorectal cancer, kidney cancer, lung cancer, lymphoma, melanoma, oral/oropharyngeal cancer, pancreatic cancer, prostate cancer, thyroid cancer, and uterine cancer.

In some embodiments, a disease, disorder, or condition is a genetic disorder. In some embodiments, a genetic disorder is associated with a gain-of-function mutation or a loss-of-function mutation.

In some embodiments, a disease, disorder, or condition is an autoimmune disease. In some embodiments, an autoimmune disease is selected from addison disease, celiac disease, rheumatoid arthritis, lupus, inflammatory bowel disease, dermatomyositis, multiple sclerosis, diabetes, guillain-barre syndrome, chronic inflammatory demyelinating polyneuropathy, psoriasis, pernicious anemia, graves' disease, hashimoto's thyroiditis, myasthenia gravis, and vasculitis sjörgen syndrome.

In some embodiments, a disease, disorder, or condition is a rare disease. As described herein, a rare disease refers to a life-threatening or chronically debilitating diseases which are of such low prevalence (e.g., fewer than 1/2000 people) that special combined efforts are needed to address them.

In some embodiments, the present disclosure provides complexes that can selectively target particular systems within a body. As used herein, reference to “targeting” a particular system refers to causing increased expression of RNA derived from cargo in the complex in the desired system. For example, in some embodiments, complexes described herein can selectively target the lungs, liver, spleen, heart, brain, lymph nodes, bladder, kidneys, and pancreas. As described herein, a complex “selectively targets” an organ when a single target expresses mRNA in an amount that is 65% or greater than expression in other organs post administration (e.g., 65% or more of mRNA throughout the body is expressed from a single organ, with the remaining 35% distributed between one or more different organs). In some embodiments, a complex described herein selectively targets the lungs. In some embodiments, a complex described herein selectively targets the liver. In some embodiments, a complex described herein selectively targets the spleen. In some embodiments, a complex described herein selectively targets the heart.

### EXEMPLARY EMBODIMENTS

The following numbered embodiments, while non-limiting, are exemplary of certain aspects of the present disclosure:

Embodiment 1. A complex comprising a first cationic polymer, a second cationic polymer, and a monomeric RNA molecule, wherein the first cationic polymer comprises greater than 250 repeating monomer units, and the second cationic polymer comprises 200 repeating monomer units, or less.

Embodiment 2. The complex of Embodiment 1, wherein the first cationic polymer is or comprises poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof.

Embodiment 3. The complex of Embodiment 1 or 2, wherein the first cationic polymer is a homopolymer selected from poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof.

Embodiment 4 The complex of Embodiments 2 or 3, wherein the first cationic polymer is linear poly(ethylenimine).

Embodiment 5. The complex of Embodiment 4, wherein the linear poly(ethylenimine) has a Mₙ of about 600 Da to about 400,000 Da.

Embodiment 6. The complex of Embodiment 5, wherein the linear poly(ethylenimine) has a Mₙ of about 10,000 Da to about 120,000 Da.

Embodiment 7. The complex of any one of Embodiments 1 or 2, wherein the first cationic polymer is a linear block-co-polymer comprising poly(ethylenimine) and poly(propylenimine).

Embodiment 8. The complex of Embodiment 1, wherein the first cationic polymer is a polymer or copolymer comprising blocks of formula I and/or II:

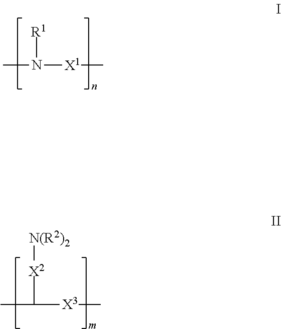

or a pharmaceutically acceptable salt thereof, wherein:
R¹ is H, —C(O)-optionally substituted C₁-C₆ aliphatic, optionally substituted C₁-C₆ aliphatic or G;
each R² is independently H, optionally substituted C₁-C₆ aliphatic, or G;
X¹ and X³ are each independently C₁-C₆ aliphatic;
X² is a bond or optionally substituted C₁-C₆ aliphatic;
m is an integer between 2 and 2000;
n is an integer between 2 and 2000;
each G is independently:

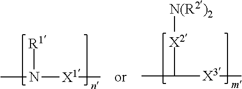

R^{1′} is H, —C(O)-optionally substituted C₁-C₆ aliphatic, optionally substituted C₁-C₆ aliphatic, or G′;
each R^{2′} is independently H, optionally substituted C₁-C₆ aliphatic, or G′;
X^{1′} and X^{3′} are each independently optionally substituted C₁-C₆ aliphatic;
X^{2′} is a bond or optionally substituted C₁-C₆ aliphatic;
n′ is an integer between 2 and 2000;
m′ is an integer between 2 and 2000;
each G′ is independently:

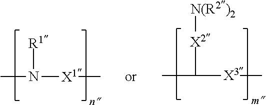

R^{1″} is H, —C(O)-optionally substituted C₁-C₆ aliphatic, or optionally substituted C₁-C₆ aliphatic;
each R^{2″} is independently H or C₁-C₆ aliphatic;
X^{1″} and X^{3″} are each independently optionally substituted C₁-C₆ aliphatic;
X^{2″} is a bond or optionally substituted C₁-C₆ aliphatic;
n″ is an integer between 2 and 2000; and
m″ is an integer between 2 and 2000.

Embodiment 9. The complex of any one of Embodiments 1-8, wherein the second cationic polymer is or comprises a polypeptide, a peptide, a protein, a polysaccharide, or a polyimine.

Embodiment 10. The complex of Embodiment 9, wherein the second cationic polymer is a polypeptide having 25% or more monomers selected from arginine, histidine, lysine, and a non-natural amino acid.

Embodiment 11. The complex of Embodiment 10, wherein the peptide is a TAT peptide, a TAT-derived peptide, poly-L-arginine or a poly-L-arginine-derived peptide.

Embodiment 12. The complex of Embodiment 9, wherein the polysaccharide is chitosan or diethylaminoethyl dextran.

Embodiment 13. The complex of Embodiment 9, wherein the polyimine is poly(ethylenimine), poly(propylenimine), poly(amidoamine), polyaminoester, poly(allylamine), polyvinylamine, poly(dimethylaminoethyl methacrylate), or polybrene.

Embodiment 14. The complex of any one of Embodiments 1-13, wherein the second cationic polymer comprises less than 200 monomeric units.

Embodiment 15. The complex of any one of Embodiments 1-14, wherein the second cationic polymer comprises 100 monomeric units or less.

Embodiment 16. The complex of any one of Embodiments 1-15, wherein the second cationic polymer comprises 30 monomeric units or less.

Embodiment 17. The complex of any one of Embodiments 1-16, wherein the second cationic polymer comprises about 20 monomeric units.

Embodiment 18. The complex of any one of Embodiments 1-17, wherein a molar ratio of the first cationic polymer to the second cationic polymer in the complex is from about 50:50 to about 5:95.

Embodiment 19. The complex of Embodiment 18, wherein the molar ratio of the first cationic polymer to the second cationic polymer in the complex is from about 25:75 to about 12.5:87.5. Embodiment 20. The complex of any one of Embodiments 1-19, further comprising a buffering agent.

Embodiment 21. The complex of Embodiment 20, wherein the buffering agent is selected from 4-(2-hydroxyethyl)-1-piperazineethanesulfonic acid (HEPES), 3-morpholino-2-hydroxypropanesulfonic acid (MOPSO), 2-(N-morpholino) ethanesulfonic acid (MES), 2-Amino-2 (hydroxymethyl) propane-1,3-diol (TRIS), Bis-tris buffering systems, carboxylic acid buffering systems, phosphatic acid buffering systems, or citric acid buffering systems.

Embodiment 22. The complex of any one of Embodiments 1-21, wherein the monomeric RNA molecule is mRNA, modRNA, saRNA, or taRNA.

Embodiment 23. The complex of any one of Embodiments 1-22, wherein the complex has a diameter of about 10 nm to about 70 nm.

Embodiment 24. The complex of any one of Embodiments 1-23, wherein the complex has a diameter of about 20 nm to about 50 nm.

Embodiment 25. A method of increasing or causing increased expression of RNA in a target in a subject comprising administering to the subject the complex of any one of Embodiments 1-24.

Embodiment 26. The method of Embodiment 25, wherein the target is selected from the lungs, liver, spleen, heart, brain, lymph nodes, bladder, kidneys, and pancreas.

Embodiment 27. A method of treating a disease, disorder, or condition in a subject comprising administering to the subject a complex of any one of Embodiments 1-24.

Embodiment 28. The method of Embodiment 27, wherein the disease, disorder, or condition is an infectious disease, cancer, a genetic disorder, an autoimmune disease, or a rare disease.

Embodiment 29. The method of any one of Embodiments 25-28, wherein the complex is administered parenterally.

Embodiment 30. The method of Embodiment 29, wherein the complex is administered intramuscularly, subcutaneously, or intravenously.

Embodiment 31. The method of any one of Embodiments 25-28, wherein the complex is administered intranasally.

Embodiment 32. The method of any one of Embodiments 25-28, wherein a first dose of the complex is administered by a first route, and a second dose of the complex is administered by a second route.

Embodiment 33. The method of Embodiment 32, wherein the first dose is administered by parenteral means.

Embodiment 34. The method of Embodiments 32 or 33, wherein the second dose is administered by intranasal means.

Embodiment 35. A complex of any one of Embodiments 1-24, for use as a medicament.

Embodiment 36. A complex of any one of Embodiments 1-24, for use in the treatment and/or prevention of a disease, disorder, or condition, wherein the disease, disorder, or condition is an infectious disease, cancer, a genetic disorder, an autoimmune disease, or a rare disease.

### EXAMPLES

As described in the Examples below, in certain exemplary embodiments, compounds are prepared according to the following general procedures. It will be appreciated that, although the general methods depict the synthesis of certain compounds of the present disclosure, the following general methods and other methods known to one of ordinary skill in the art can be applied to all compounds and subclasses and species of each of these compounds, as described herein.

Table of Abbreviations

| | |
|---|---|
| Da | Dalton |
| DP | Degree of polymerization |
| i.m. | Intramuscular |
| MBG | MES buffered glucose |
| MES | 2-(N-morpholino)ethanesulfonic acid |
| pArg | poly-L-arginine |
| PEI | Polyethylenimine |

### Example 1—Use of a TAT-Derived Polypeptide in a Complex

The present example reports properties of complexes prepared with polyethylenimine and a TAT-derived polypeptide using saRNA or modRNA at N/P12 in MBG Buffer (final concentration 5% w/v Glucose, 10 mM MES, pH 6.1) and at RNA final concentration of 0.1 mg/ml, where the mixing ratio of the RNA containing solution and the cationic polymers mix containing solution was 99:1 (v:v).

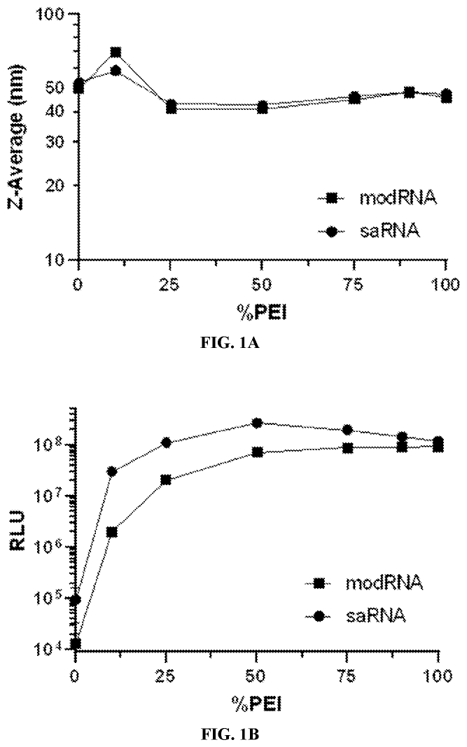

The tested complexes comprised linear polyethylenimine (500DP or 22.5 kDa, hereinafter “PEI”) and a modified version of TAT (CHHHHHRKKRRQRRRRHHHHHC) (SEQ ID NO: 2), referred to as “mTAT”. A total of seven different mixing ratios of PEI-mTAT are tested. In FIGS. 1A and 1B, the percentage of PEI (% PEI) represents the proportional amount of cationic charges in the polymer mix that are coming from PEI polymer, as determined by molar % (i.e., 50% PEI refers to 50% of cationic charges coming from PEI, with the remaining 50% coming from mTAT). The total amount of charges in the polymer mix are kept constant between all the groups. FIG. 1A is a plot illustrating the average hydrodynamic diameter of formulated with saRNA or modRNA at different PEI-mTAT mixing ratios. FIG. 1B is a plot illustrating luciferase expression of complexes formulated with saRNA or modRNA at different PEI-mTAT mixing ratios in C2C12 cells and a 100 ng dose.

### Example 2—Use of Poly-L-Arginine in a Complex

The present example reports properties of complexes prepared with polyethylenimine and a poly-L-arginine (pArg) using saRNA or modRNA at N/P12 in MBG Buffer (final concentration 5% w/v Glucose, 10 mM MES, pH 6.1) and at RNA final concentration of 0.1 mg/ml, where the mixing ratio of the RNA containing solution and the cationic polymers mix containing solution was 99:1 (v:v).

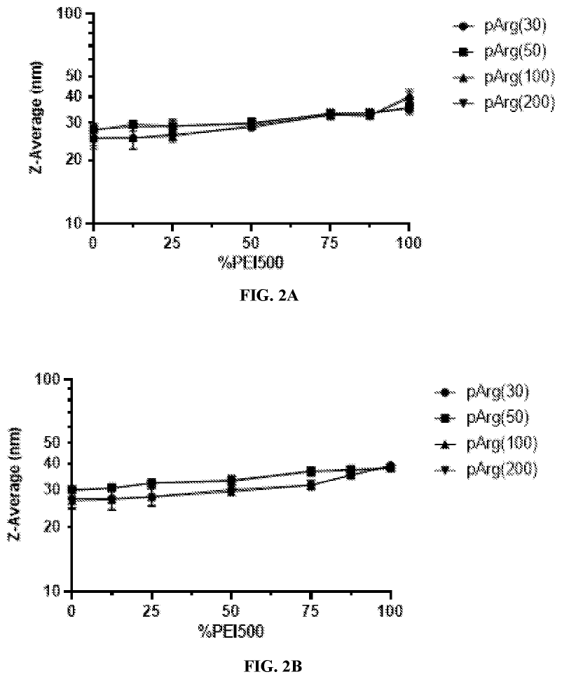

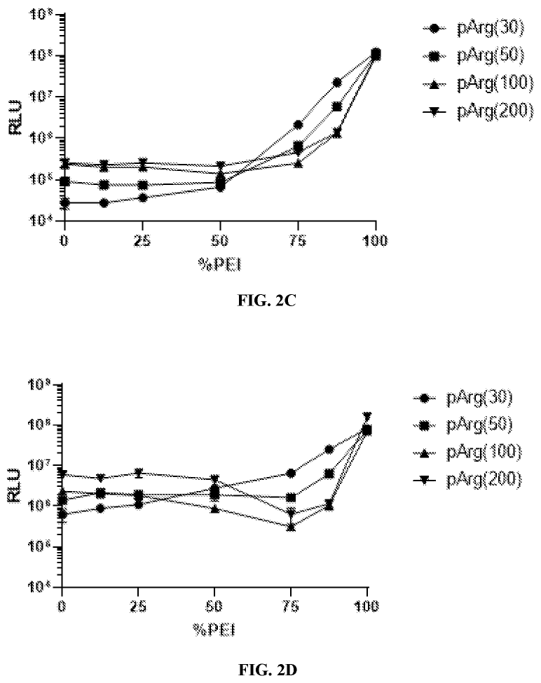

The tested complexes comprised linear polyethylenimine (500DP or 22.5 kDa, hereinafter PEI) and poly-L-Arginine of different lengths (30, 50, 100, or 200 monomer units, referred to as pArg(30), pArg(50), pArg(100), pArg(200), respectively). A total of seven different mixing ratios of PEI-pArg are tested. In FIGS. 2A-2D, the percentage of PEI (% PEI) represents the proportional amount of cationic charges in the polymer mix that are coming from PEI polymer, as determined by molar % (i.e., 50% PEI refers to 50% of cationic charges coming from PEI, with the remaining 50% coming from pArg). The total amount of charges in the polymer mix are kept constant between all the groups. FIG. 2A is a plot illustrating the average hydrodynamic diameter of complexes comprising modRNA and having different PEI-pArg mixing ratios and pArg lengths. FIG. 2B is a plot illustrating the average hydrodynamic diameter of complexes comprising saRNA and having different PEI-pArg mixing ratios and pArg lengths. FIG. 2C is a plot illustrating the luciferase expression of complexes comprising modRNA and having different PEI-pArg mixing ratios and pArg lengths in C2C12 cells at a 100 ng dose. FIG. 2D is a plot illustrating the luciferase expression of complexes comprising saRNA having different PEI-pArg mixing ratios and pArg lengths in C2C12 cells at a 100 ng dose.

### Example 3—In Vivo Administration of a Complex Comprising mTAT

The present example reports properties of complexes prepared with polyethylenimine and a modified TAT peptide using saRNA or modRNA at N/P12 in MBG Buffer (final concentration 5% w/v Glucose, 10 mM MES, pH 6.1) and at RNA final concentration of 0.1 mg/ml, where the mixing ratio of the RNA containing solution and the cationic polymers mix containing solution was 99:1 (v:v).

The tested complexes comprised linear polyethylenimine (500DP or 22.5 kDa) and a modified version of TAT (CHHHHHRKKRRQRRRRHHHHHC) (SEQ ID NO:2), referred to as “mTAT”. Seven different complexes comprising PEI-mTAT were tested. The percentage of PEI (% PEI) represents the proportional amount of cationic charges in the polymer mix that are coming from PEI polymer, as determined by molar % (i.e., 50% PEI refers to 50% of cationic charges coming from PEI, with the remaining 50% coming from mTAT). The total amount of charges in the polymer mix are kept constant between all the groups.

The complexes were tested in BALB/c mice. Twenty seven mice were divided into 9 groups. All groups received 2 μg of RNA, by intramuscular (i.m.) administration, either modRNA or saRNA, formulated according to the table below:

| Complex | PET %-mTAT % | RNA |
|---|---|---|
| 1 | 100%-0% | saRNA |
| 2 | 50%-50% | saRNA |
| 3 | 25%-75% | saRNA |
| 4 | 12.5%-87.5% | saRNA |
| 5 | 100%-0% | modRNA |
| 6 | 50%-50% | modRNA |
| 7 | 25%-75% | modRNA |
| 8 | 12.5%-87.5% | modRNA |
| 9 | 0-100% | modRNA |

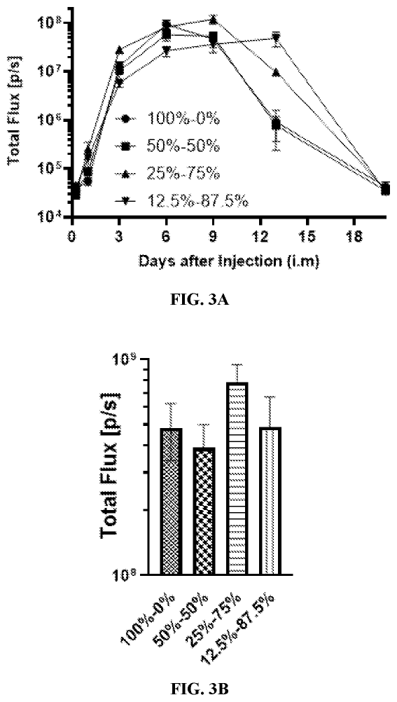

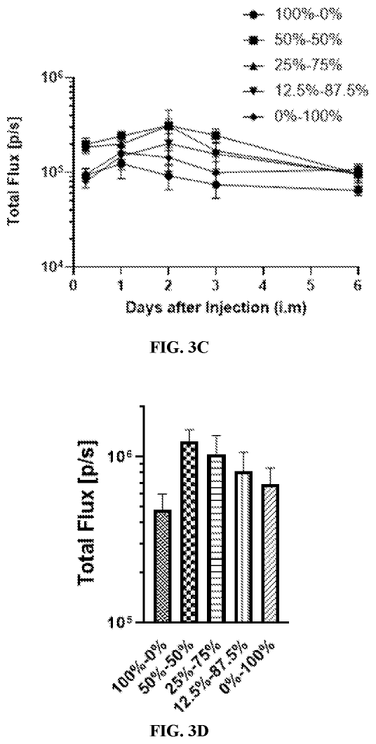

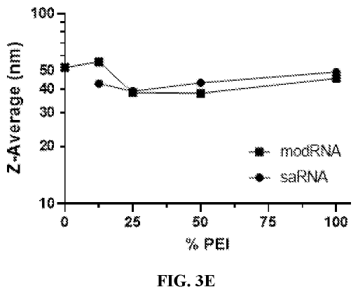

FIG. 3A is a plot illustrating luciferase expression for the tested complexes over the course of the experiment with formulated saRNA at different PEI-mTAT ratios. FIG. 3B is a plot illustrating the area under the curve of luciferase expression over the whole duration of the experiment with formulated saRNA at different PEI-mTAT mixing ratios. FIG. 3C is a plot illustrating luciferase expression for the tested complexes over time. FIG. 3D is a plot illustrating the area under the curve of luciferase expression over time with tested complexes. FIG. 3E is a plot illustrating the average hydrodynamic diameter of the tested complexes.

1. A complex comprising a first cationic polymer, a second cationic polymer, and a monomeric RNA molecule, wherein the first cationic polymer comprises greater than 250 repeating monomer units, and the second cationic polymer comprises 200 repeating monomer units, or less.

2. The complex of claim 1, wherein the first cationic polymer is or comprises poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof.

3. The complex of claim 1 or 2, wherein the first cationic polymer is a homopolymer selected from poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof.

4. The complex of claim 2 or 3, wherein the first cationic polymer is linear poly(ethylenimine).

5. The complex of claim 4, wherein the linear poly(ethylenimine) has a Mₙ of about 600 Da to about 400,000 Da.

6. The complex of claim 5, wherein the linear poly(ethylenimine) has a Mₙ of about 10,000 Da to about 120,000 Da.

7. The complex of any one of claim 1 or 2, wherein the first cationic polymer is a linear block-co-polymer comprising poly(ethylenimine) and poly(propylenimine).

8. The complex of claim 1, wherein the first cationic polymer is a polymer or copolymer comprising blocks of formula I and/or II: 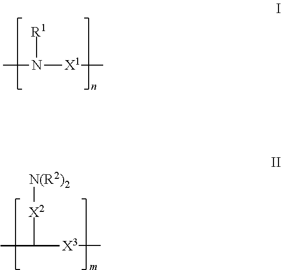
or a pharmaceutically acceptable salt thereof, wherein:
R¹ is H, —C(O)-optionally substituted C₁-C₆ aliphatic, optionally substituted C₁-C₆ aliphatic or G;
each R² is independently H, optionally substituted C₁-C₆ aliphatic, or G;
X¹ and X³ are each independently C₁-C₆ aliphatic;
X² is a bond or optionally substituted C₁-C₆ aliphatic;
m is an integer between 2 and 2000;
n is an integer between 2 and 2000;
each G is independently: 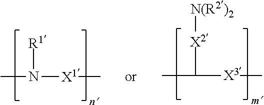
R^{1′} is H, —C(O)-optionally substituted C₁-C₆ aliphatic, optionally substituted C₁-C₆ aliphatic, or G′;
each R^{2′} is independently H, optionally substituted C₁-C₆ aliphatic, or G′;
X^{1′} and X^{3′} are each independently optionally substituted C₁-C₆ aliphatic;
X^{2′} is a bond or optionally substituted C₁-C₆ aliphatic;
n′ is an integer between 2 and 2000;
m′ is an integer between 2 and 2000;
each G′ is independently: 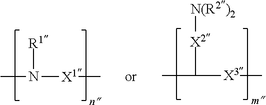
R¹″ is H, —C(O)-optionally substituted C₁-C₆ aliphatic, or optionally substituted C₁-C₆ aliphatic;
each R^{2″} is independently H or C₁-C₆ aliphatic;
X^{1″} and X^{3″} are each independently optionally substituted C₁-C₆ aliphatic;
X^{2″} is a bond or optionally substituted C₁-C₆ aliphatic;
n″ is an integer between 2 and 2000; and
m″ is an integer between 2 and 2000.

9. The complex of any one of claims 1-8, wherein the second cationic polymer is or comprises a polypeptide, a peptide, a protein, a polysaccharide, or a polyimine.

10. The complex of claim 9, wherein the second cationic polymer is a polypeptide having 25% or more monomers selected from arginine, histidine, lysine, and a non-natural amino acid.

11. The complex of claim 10, wherein the peptide is a TAT peptide, a TAT-derived peptide, poly-L-arginine or a poly-L-arginine-derived peptide.

12. The complex of claim 9, wherein the polysaccharide is chitosan or diethylaminoethyl dextran.

13. The complex of claim 9, wherein the polyimine is poly(ethylenimine), poly(propylenimine), poly(amidoamine), polyaminoester, poly(allylamine), polyvinylamine, poly(dimethylaminoethyl methacrylate), or polybrene.

14. The complex of any one of claims 1-13, wherein the second cationic polymer comprises less than 200 monomeric units.

15. The complex of any one of claims 1-14, wherein the second cationic polymer comprises 100 monomeric units or less.

16. The complex of any one of claims 1-15, wherein the second cationic polymer comprises 30 monomeric units or less.

17. The complex of any one of claims 1-16, wherein the second cationic polymer comprises about 20 monomeric units.

18. The complex of any one of claims 1-17, wherein a molar ratio of the first cationic polymer to the second cationic polymer in the complex is from about 50:50 to about 5:95.

19. The complex of claim 18, wherein the molar ratio of the first cationic polymer to the second cationic polymer in the complex is from about 25:75 to about 12.5:87.5.

20. The complex of any one of claims 1-19, further comprising a buffering agent.

21. The complex of claim 20, wherein the buffering agent is selected from 4-(2-hydroxyethyl)-1-piperazineethanesulfonic acid (HEPES), 3-morpholino-2-hydroxypropanesulfonic acid (MOPSO), 2-(N-morpholino)ethanesulfonic acid (MES), 2-Amino-2(hydroxymethyl)propane-1,3-diol (TRIS), Bis-tris buffering systems, carboxylic acid buffering systems, phosphatic acid buffering systems, or citric acid buffering systems.

22. The complex of any one of claims 1-21, wherein the monomeric RNA molecule is mRNA, modRNA, saRNA, or taRNA.

23. The complex of any one of claims 1-22, wherein the complex has a diameter of about 10 nm to about 70 nm.

24. The complex of any one of claims 1-23, wherein the complex has a diameter of about 20 nm to about 50 nm.

25. A method of increasing or causing increased expression of RNA in a target in a subject comprising administering to the subject the complex of any one of claims 1-24.

26. The method of claim 25, wherein the target is selected from the lungs, liver, spleen, heart, brain, lymph nodes, bladder, kidneys, and pancreas.

27. A method of treating a disease, disorder, or condition in a subject comprising administering to the subject a complex of any one of claims 1-24.

28. The method of claim 27, wherein the disease, disorder, or condition is an infectious disease, cancer, a genetic disorder, an autoimmune disease, or a rare disease.

29. The method of any one of claims 25-28, wherein the complex is administered parenterally.

30. The method of claim 29, wherein the complex is administered intramuscularly, subcutaneously, or intravenously.

31. The method of any one of claims 25-28, wherein the complex is administered intranasally.

32. The method of any one of claims 25-28, wherein a first dose of the complex is administered by a first route, and a second dose of the complex is administered by a second route.

33. The method of claim 32, wherein the first dose is administered by parenteral means.

34. The method of claim 32 or 33, wherein the second dose is administered by intranasal means.

35. A complex of any one of claims 1-24, for use as a medicament.

36. A complex of any one of claims 1-24, for use in the treatment and/or prevention of a disease, disorder, or condition, wherein the disease, disorder, or condition is an infectious disease, cancer, a genetic disorder, an autoimmune disease, or a rare disease.


SEQUENCE LISTING (2 sequences)

SEQ ID NO: 1 - protein, 12 aa, synthetic construct
GRKKRRQRRRPQ

SEQ ID NO: 2 - protein, 22 aa, synthetic construct
CHHHHHRKKRRQRRRRHHHHHC